(19) United States
(12) Patent Application Publication
(10) Pub. No.: US 2024/0325571 A1
(43) Pub. Date: Oct. 3, 2024

(54) COMPOSITIONS AND METHODS FOR TREATING INFLAMMATORY DISEASE

(71) Applicant: Christopher Key, Orange, CA (US)

(72) Inventor: Christopher Key, Orange, CA (US)

(21) Appl. No.: 18/739,865

(22) Filed: Jun. 11, 2024

Related U.S. Application Data
(63) Continuation of application No. 17/708,911, filed on Mar. 30, 2022, now Pat. No. 12,011,489.

(51) Int. Cl. A61K 48/00; A61K 35/76; A61K 35/761; A61K 38/17

(52) U.S. Cl. CPC A61K 48/0066; A61K 35/76; A61K 35/761; A61K 38/177

(57) ABSTRACT

Compositions and methods are provided that mitigate iron imbalance resulting from inflammation and/or mitigate the effects of inflammatory disease by correcting dysregulation of iron internalization. Such correction is provided by modulating cellular content of TFR2, and/or hepcidin activity in an individual in need of treatment. TRF2 content can be modulated by correcting a mutation in an endogenous TRF2 gene of the individual.

This application is a continuation-in-part of U.S. patent application Ser. No. 17/708,911 filed on Mar. 30, 2022. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is treatment of inflammation-related diseases and conditions.

### BACKGROUND

The following description includes information that may be useful in understanding the present invention. It is not an admission that any of the information provided herein is prior art or relevant to the presently claimed invention, or that any publication specifically or implicitly referenced is prior art.

Iron is an essential nutrient that is involved in oxygen transport and acts as a cofactor in a wide range of metabolic processes and redox reactions within the cell. Iron concentration within the fluids and tissues of the body must be carefully regulated, however, as it can generate reactive oxygen species that can result in cellular damage and death. This balance between iron nutrition and toxicity is provided by systemic control mechanisms that modulate iron conservation and uptake. This is in contrast with regulation of other metals, which are controlled by simply eliminating excess. Iron and its homeostasis are closely connected with the response to inflammation and infection, and therefore are major survival mechanisms.

A large body of evidence shows that susceptibility to disease as well as response to infection and inflammation worsen with iron overload. The relationships between iron overload and infectious diseases (e.g., tuberculosis, malaria) is well documented. Iron overload resulting from hereditary hemochromatosis has been found to increase susceptibility to infectious disease. Increased susceptibility to infectious disease is also found in thalassemic patients with iron overload resulting from frequent blood transfusions. Iron status has also been found to influence the course of viral infections.

Iron status also influences the course of chronic inflammatory disease. An increase in iron stores correlates with markers of chronic inflammation in the development and progression of diabetes, obesity, and metabolic syndrome. High iron load is also involved in the development and course of neurodegenerative disease. This is significant, as not only does the prevalence of chronic diseases increase as the population ages, but also because modified dietary iron or manipulation of iron status could represent simple preventive or therapeutic approaches.

Typically, high iron stores are reduced by phlebotomy or iron chelation therapy. While such approaches have been found to provide a degree of improvement, they do not address other effects of iron removal on necessary metabolic and energy-generating redox reactions in cells of individuals treated in this manner.

U.S. Pat. No. 9,782,453, to Nicolas et al., describes the use of hepcidin to reduce iron overload, citing observation of high tissue concentrations of iron in mice with genetic defects that reduce hepcidin expression. All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. It is not clear, however, if application of hepcidin is useful in the treatment of individuals without such a genetic defect.

United States Patent Application Publication No. 2021/0214456, to Rauner et al., describes modulating activity of transferrin receptor 2 to treat a variety of conditions. Specifically, Rauner et al. describe the use of transferrin receptor 2 inhibitors to treat anemia (such as anemia related to chronic inflammation). Use of agonists to increase transferrin receptor 2 activity, however, is limited to treatment of bone disease.

Thus, there is still a need for safe and effective methods for reducing elevated iron level in tissues of the body while avoiding negative impact to iron-related functions within the cells of an individual undergoing treatment.

### SUMMARY OF THE INVENTION

The inventive subject matter provides compositions and methods for treating inflammatory disease by modulating dysregulation of iron transport into cells of an individual in need of treatment for inflammatory disease.

Embodiments of the inventive concept include methods of treating an inflammatory disease or iron transport dysregulation resulting from inflammation by identifying an individual in need of treatment for such an inflammatory disease associated with dysregulation of iron metabolism or iron transport dysregulation resulting from inflammation; and modifying a cell in the individual to correct mutations in a gene encoding Transferrin Receptor 2 (TFR2). Such mutations include point mutations (e.g., to a codon for an incorrect amino acid or to a stop codon) and deletions. In some embodiments the TRF2 gene within the genome is modified using a suitable gene editing tool to correct the mutation (e.g., to revert the TRF2 gene to a wild type sequence) or replace the genomic TRF2 gene. In other embodiments an exogenous TRF2 gene is incorporated into the individual nuclear DNA or into an organelle. In some embodiments such methods can include providing a supplemental iron removal therapy to the individual, such as phlebotomy and/or chelation therapy.

Some embodiments of the inventive concept is a methods of treating an inflammatory disease by identifying an individual in need of treatment for an inflammatory disease associated with dysregulation of iron metabolism, and modifying one or more cell(s) of the individual to up-regulate expression of hepcidin or increase hepcidin content of a cell of an individual so afflicted. Examples of such diseases include Parkinson Disease, Alzheimer's disease, a viral disease (e.g., infection with a coronavirus such as SARS-COV-2), and inflammatory response to a pathogen. The TFR2 can include alpha Transferrin Receptor 2 (αTFR2) and/or beta Transferrin Receptor 2 (βTFR2). In some embodiments the method includes introducing an expression vector that encodes hepcidin. In other embodiments the method includes introducing an expression vector that encodes a factor that interacts with a regulatory element associated with hepcidin. In such methods the expression vector can be a virus, such as a genetically modified influenza virus or adenovirus, and can include an inducible promoter. In some embodiments the method includes delivering hepcidin to a cell of the individual, for example as hepcidin enclosed in a micelle or similar membrane. In some embodiments the method can also include a supplemental iron removal therapy, such as phlebotomy and chelation therapy.

Embodiments of the inventive concept include compositions for treating an inflammatory disease or dysregulation of iron transport resulting from inflammation, for example in methods such as described above. Such compositions include a micelle or vesicle that encloses TRF2, wherein TRF2 includes at least one of αTFR2 protein or an active fragment thereof and βTFR2 protein or an active fragment thereof, or hepcidin. In some embodiments suitable compositions include a virus (such as an adenovirus) that incorporates components of a gene editing composition suitable for replacing all or a portion of an individual's endogenous TRF2 gene. In some embodiments suitable compositions include a virus (such as an adenovirus or a retrovirus) that incorporates an exogenous TRF2 gene.

Various objects, features, aspects and advantages of the inventive subject matter will become more apparent from the following detailed description of preferred embodiments.

### DETAILED DESCRIPTION

The presence of high circulating concentration of iron (i.e., in the form of ferritin) is frequently associated with inflammatory processes. Typically, this is treated using therapies that deplete the body's iron stores (e.g., phlebotomy) or that complex circulating iron (e.g., chelation therapy). The Inventor contemplates an alternative approach, in which high circulating concentrations of iron associated with inflammatory processes are a result of dysregulation of internalization of iron by cells of the body, which can result in elevated concentrations of circulating iron and dysfunction of iron-related processes (e.g., mitochondrial production of ATP) within cells of the body due to inadequate iron within the cells. This can be addressed by increasing cellular iron internalization, for example by upregulating expression or otherwise increasing the content of proteins of the cell that are involved in iron internalization. Such proteins include, but are not limited to, transferrin receptor 2 (TFR2) and forms thereof (e.g., αTFR2, βTFR2). As a result, circulating iron concentrations are reduced and iron-related metabolic processes in cells of the body are corrected.

One should appreciate that the reduction in circulating iron concentrations coupled with increased cellular internalization of iron, with a concomitant reduction in inflammation or at least some of the deleterious effects of inflammation can reduce symptoms and improving outcome in a wide variety and inflammatory and inflammation-related diseases and conditions.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The Inventor believes that the association between serum iron concentration and inflammation (and diseases and/or conditions resulting from such inflammation) can be understood, at least in part, as a dysregulation of iron transport into cells of the human body. For example, excessive circulating iron (e.g., ferritin in the bloodstream) can result in impaired transport of iron into tissues and/or cells of the body. Specifically, such effects can result in insufficient transport of iron into cells, which in turn negatively impacts mitochondrial function. Iron is utilized as a cofactor in mitochondrial processes that provide ATP and other molecules necessary for normal cell function. The Inventor believes that elevated serum iron concentrations (for example, as a result of an inflammatory condition) can cause or at least in part result from of a dysregulation or impairment of iron transport into the cells. Such dysregulation can result in or be a result of elevated iron concentration in serum, which can in turn lead to sub-optimal iron concentration within the cells (e.g., due to iron being dysregulated, cells' mitochondrial function can be impaired). This impairment of mitochondrial function impairs cell function, resulting in the development or exacerbation of disease in the individual. Such exacerbation can contribute to the effects of inflammatory disease.

Accordingly, correction or even partial correction of such dysregulation of iron transport into the cell can act to cure or at least partially alleviate diseases and conditions associated with inflammation related to dysregulation of iron transport. Similarly, correction or even partial correction of such dysregulation of iron transport into the cell can act to restore more normal cellular function and mitigate the effects of an inflammatory condition and/or improve the effects of conventional treatment for an inflammatory condition. Such correction can be achieved, for example, by increasing iron transport into the cells.

One embodiment of the inventive concept is a method of treating or mitigating the effects of an inflammatory disease by modifying cells of an individual with or at risk of the inflammatory disease to increase their uptake of iron. Examples of inflammatory disease that can be treated include, but are not limited to, Alzheimer's disease, Parkinson disease, Behcet's disease, neurodegenerative disease, heart disease, diabetes, cancer, arthritis, ankylosing spondylitis, asthma, type 2 diabetes, an inflammatory response to infection (e.g., viral, bacterial, fungal, or protozoan infection), and infection with a coronavirus (e.g., SARS-COV-2).

In some embodiments correction of the dysregulation of iron transport into cells can be utilized to mitigate damage to tissues and/or cells of the body resulting from inflammatory diseases or processes. For example, damage to tissues and/or cells of the body can be reduced or eliminated as underlying and treatable causes of inflammation are addressed. Examples of such underlying and treatable causes of inflammation include poor diet, vitamin deficiency, obesity, stress, and infection.

An initial step in such a method can be identifying an individual that has or is at risk of developing such an inflammatory disease. This can be accomplished by any suitable means. For example, such an individual can be identified on the basis of current symptoms, medical history, and/or laboratory results indicative of dysregulation of iron concentration and/or the inflammatory disease. In some embodiments such an individual can be identified on the basis of their genetics and/or family history. For example, an individual can be identified as having or being at risk of developing such an inflammatory disease based on DNA sequencing, characterization of their RNA expression, characterization of their proteome, genetic analysis of relatives, and/or a family history of the inflammatory disease and/or dysregulation of iron concentration. In some embodiments the individual in need of treatment is symptomatic. In other embodiments the individual in need of treatment is asymptomatic. In some embodiments of the inventive subject matter the individual in need of treatment shows a genetic predisposition towards development of an inflammatory disease related to iron dysregulation (e.g., by family history, genetic studies, etc.) but shows no indication of having yet developed the disease. In such a case the individual's treatment can be considered prophylactic.

In some embodiments an individual in need of treatment can be identified by characterizing efficiency in internalization of circulating iron into their tissues. For example, circulating iron concentration can be characterized by measuring the concentration of circulating ferritin, whereas tissue and/or cellular uptake of iron can be characterized by measuring iron content of a tissue sample obtained from the patient. Significant deviation from mean or median values derived from a normal or healthy population (e.g., a 5%, 10%, 15%, 20%, 25%, 35%, 50%, 75%, 90%, or more reduction in a tissue or cellular iron content to circulating iron content ratio) can be considered indicative of impaired iron internalization. Alternatively, tissue or cellular iron content can be similarly compared to those of normal or healthy individuals to determine if overall iron internalization is abnormal.

On identifying an individual in need of treatment for an inflammatory disease associated with dysregulation of iron metabolism and/or iron dysregulation as a result of inflammation, the individual can be treated to increase cellular iron uptake. In some embodiments this can be accomplished by modifying a cell of the individual to up-regulate expression of Transferrin Receptor 2 (TFR2). It should be appreciated that TFR2 occurs in different forms, and that such up-regulation can be directed to alpha Transferrin Receptor 2 (αTFR2), beta Transferrin Receptor 2 (βTFR2), or both.

Up-regulation of TFR2 within cells of the individual to be treated can be accomplished by any suitable means. In some embodiments such up-regulation can be accomplished by introducing an expression vector that encodes αTFR2, βTFR2, or both αTFR2 and βTFR2 into cells. Alternatively, such up-regulation can be accomplished by introducing an expression vector that encodes a factor that interacts with a regulatory element located upstream from an endogenous or native gene encoding αTFR2, βTFR2, or both αTFR2 and βTFR2, where such interaction results in increased expression of the endogenous or native gene. The resulting increase in TFR2 content in the modified cell can act to increase transport of iron from the surrounding environment into the cell, thereby improving or restoring mitochondrial function while decreasing serum iron concentrations.

Such an expression vector can take any suitable form. In some embodiments the expression vector is a genetically modified virus. Suitable viruses include retroviruses, herpes viruses, pox viruses, influenza viruses, and adenoviruses. Such viruses can be modified, for example by genetic modification and/or attenuation, to reduce virulence. For example, virulence of a pox virus utilized as a vector of the inventive concept can be reduced by attenuation through repeated passage through non-host species cells in culture. In some embodiments a viral vector can be used that is known to be rapidly cleared by the immune system. In other embodiments a viral vector can be used that produces a persistent infection or that modifies DNA of the host cell in order to provide a persistent effect.

Alternatively, in some embodiments an expression vector can be provided in a micelle, liposome, or similar synthetic membrane package. Such a micelle or liposome can incorporate proteins, sugars, or other biomolecules into the exposed surface in order to facilitate targeting of and/or incorporation of cells of the individual being treated. For example, such a micelle or lipid can enclose a nucleic acid encoding for TFR2 and/or a factor that upregulates endogenous TFR2 expression and carry receptors for cell surface markers characteristic of target cells within the individual to be treated.

In some embodiments the expression vector can include a promoter positioned upstream of an encoding region that encodes for αTFR2, βTFR2, both αTFR2 and βTFR2, or a regulatory factor that increases expression of endogenous αTFR2 and/or βTFR2. Such a promoter can be a promoter that is normally present in the vector being used, for example a promoter that is present in the wild-type viral genome prior to genetic modification. Alternatively, such a promoter can be introduced through genetic engineering techniques as are known in the art. In such embodiments the promoter can be inducible, thereby providing control of when the encoding region is transcribed. In such embodiments administration of the vector to the individual to be treated can be accompanied by or followed with administration of a compound that interacts with the promoter, thereby inducing transcription of the encoding region at a desired time point and/or for a desired duration.

Alternatively, TFR2 (e.g., αTFR2, βTFR2, or both αTFR2 and βTFR2) or active fragments thereof can be provided to cells as functional proteins, functional peptides, or precursors that are processed by the cell into active forms. For example, such proteins, peptides, or precursors can be provided within a micelle or liposome that can fuse with or be internalized by cells of the individual to be treated. Such a micelle or liposome can incorporate proteins, sugars, or other biomolecules into the exposed surface in order to facilitate targeting of and/or incorporation of cells of the individual being treated. For example, such a micelle or lipid can enclose TFR2, a functional fragment of TFR2, and/or a precursor of TFR2 and carry receptors for cell surface markers characteristic of target cells within the individual to be treated.

Transferrin is a glycoproteins that is produced in the liver and binds iron, mediating its transport in blood. Each molecule of transferrin contains binding sites for two Fe³⁺ ions; binding to transferrin renders these ions nontoxic. Transferrin binds iron tightly but reversibly, releasing the bound iron at the low pH encountered following complexation with transferrin receptor and subsequent transport into the cell.

In some embodiments methods of the inventive concept can include modulating transferrin content of the blood plasma in an individual in need of treatment for dysregulation of iron transport, such as those causing or resulting from inflammation. In some embodiments exogenous transferrin protein can be provided to the individual, for example by injection or infusion into the bloodstream. In such embodiments transferrin can be modified to increase serum half-life, for example by PEGylation.

In other embodiments expression of transferrin in an individual being treated can be upregulated, thereby causing an increase in the release of transferring into the bloodstream. Up-regulation of transferrin expression in the individual to be treated can be accomplished by any suitable means. In some embodiments such up-regulation can be accomplished by introducing an expression vector that encodes transferrin into cells of the individual being treated. Alternatively, such up-regulation can be accomplished by introducing an expression vector that encodes a factor that interacts with a regulatory element located upstream from an endogenous or native gene encoding transferrin, where such interaction results in increased expression of the endogenous or native gene. The resulting increase in transferrin content in individual's bloodstream can act to increase transport into the cell, thereby improving or restoring mitochondrial function while decreasing serum iron concentrations.

Such an expression vector can take any suitable form. In some embodiments the expression vector is a genetically modified virus. Suitable viruses include retroviruses, herpes viruses, pox viruses, influenza viruses, and adenoviruses. Such viruses can be modified, for example by genetic modification and/or attenuation, to reduce virulence. For example, virulence of a pox virus utilized as a vector of the inventive concept can be reduced by attenuation through repeated passage through non-host species cells in culture. In some embodiments a viral vector can be used that is known to be rapidly cleared by the immune system. In other embodiments a viral vector can be used that produces a persistent infection or that modifies DNA of the host cell in order to provide a persistent effect.

Alternatively, in some embodiments an expression vector can be provided in a micelle, liposome, or similar synthetic membrane package. Such a micelle or liposome can incorporate proteins, sugars, or other biomolecules into the exposed surface in order to facilitate targeting of and/or incorporation of cells of the individual being treated, with subsequent release of transferrin into circulation. For example, such a micelle or lipid can enclose a nucleic acid encoding for transferrin and/or a factor that upregulates endogenous transferrin expression and carry receptors for cell surface markers characteristic of target cells within the individual to be treated.

In some embodiments the expression vector can include a promoter positioned upstream of an encoding region that encodes for transferrin, or a regulatory factor that increases expression of endogenous transferrin. Such a promoter can be a promoter that is normally present in the vector being used, for example a promoter that is present in the wild-type viral genome prior to genetic modification. Alternatively, such a promoter can be introduced through genetic engineering techniques as are known in the art. In such embodiments the promoter can be inducible, thereby providing control of when the encoding region is transcribed. In such embodiments administration of the vector to the individual to be treated can be accompanied by or followed with administration of a compound that interacts with the promoter, thereby inducing transcription of the encoding region at a desired time point and/or for a desired duration.

In some embodiments methods to increase transferrin content in an individual's bloodstream can be combined, for example combining administration of transferrin via injection or infusion with administration of an expression vector encoding transferrin. In some embodiments methods to increase transferrin content in an individual can be combined with methods to increase TFR2 content or expression in cells of an individual being treated, as described above. The Applicant believes that such an increase in transferrin content in blood plasma in combination with an increase in TFR2 content of cells can provide a synergistic (i.e., greater than additive effect) in correcting dysregulation of iron transport into cells, in particular cells of an individual with an inflammatory condition.

Hepcidin is another regulator of iron metabolism, and acts by binding to the iron export channel ferroport and inhibiting its function. Such inhibition can result in iron sequestration within cells. In some embodiments of the inventive concept dysregulation of iron transport as a result of inflammation and/or inflammation resulting from dysregulation of iron transport can be at least partially corrected by increasing hepcidin expression. This can be accomplished by modifying a cell of the individual to up-regulate expression of hepcidin.

Up-regulation of hepcidin within cells of the individual to be treated can be accomplished by any suitable means. In some embodiments such up-regulation can be accomplished by introducing an expression vector that encodes hepcidin into cells. Alternatively, such up-regulation can be accomplished by introducing an expression vector that encodes a factor that interacts with a regulatory element located upstream from an endogenous or native gene encoding hepcidin, where such interaction results in increased expression of the endogenous or native gene. The resulting increase in hepcidin content in the modified cell can act to decrease loss of iron from the cell, thereby improving or restoring mitochondrial function while decreasing serum iron concentrations.

Such an expression vector can take any suitable form. In some embodiments the expression vector is a genetically modified virus. Suitable viruses include retroviruses, herpes viruses, pox viruses, influenza viruses, and adenoviruses. Such viruses can be modified, for example by genetic modification and/or attenuation, to reduce virulence. For example, virulence of a pox virus utilized as a vector of the inventive concept can be reduced by attenuation through repeated passage through non-host species cells in culture. In some embodiments a viral vector can be used that is known to be rapidly cleared by the immune system. In other embodiments a viral vector can be used that produces a persistent infection or that modifies DNA of the host cell in order to provide a persistent effect.

Alternatively, in some embodiments an expression vector can be provided in a micelle, liposome, or similar synthetic membrane package. Such a micelle or liposome can incorporate proteins, sugars, or other biomolecules into the exposed surface in order to facilitate targeting of and/or incorporation of cells of the individual being treated. For example, such a micelle or lipid can enclose a nucleic acid encoding for hepcidin and/or a factor that upregulates endogenous hepcidin expression and carry receptors for cell surface markers characteristic of target cells within the individual to be treated.

In some embodiments the expression vector can include a promoter positioned upstream of an encoding region that encodes for hepcidin, or a regulatory factor that increases expression of endogenous hepcidin. Such a promoter can be a promoter that is normally present in the vector being used, for example a promoter that is present in the wild-type viral genome prior to genetic modification. Alternatively, such a promoter can be introduced through genetic engineering techniques as are known in the art. In such embodiments the promoter can be inducible, thereby providing control of when the encoding region is transcribed. In such embodiments administration of the vector to the individual to be treated can be accompanied by or followed with administration of a compound that interacts with the promoter, thereby inducing transcription of the encoding region at a desired time point and/or for a desired duration.

Alternatively, hepcidin or active fragments thereof can be provided to cells as functional proteins, functional peptides, or precursors that are processed by the cell into active forms. For example, such proteins, peptides, or precursors can be provided within a micelle or liposome that can fuse with or be internalized by cells of the individual to be treated. Such a micelle or liposome can incorporate proteins, sugars, or other biomolecules into the exposed surface in order to facilitate targeting of and/or incorporation of cells of the individual being treated. For example, such a micelle or lipid can enclose hepcidin, a functional fragment of hepcidin, and/or a precursor of hepcidin and carry receptors for cell surface markers characteristic of target cells within the individual to be treated.

In some embodiments methods to increase hepcidin content in an individual's cells can be combined with methods to increase TFR2 content or expression in cells of an individual being treated and/or increasing transferrin content in the individual's bloodstream, as described above. The Applicant believes that such an increase in cellular hepcidin content in combination with an increase in TFR2 content of cells and/or transferrin content in blood plasma can provide a synergistic (i.e., greater than additive effect) in correcting dysregulation of iron transport into cells, in particular cells of an individual with an inflammatory condition.

An individual having inflammation related to iron storage dysregulation may have a mutation in the gene within their genome encodes for TRF2 (i.e., an endogenous gene) the. Such an endogenous TRF2 gene is distinct from a gene encoding TRF2 as part of an expression vector that is introduced into the individual as described above (i.e. an exogenous TRF2 gene). The Inventor contemplates that mutations within an endogenous TRF2 gene (e.g., deletions, translocation, point mutations, etc.), as an inherited condition and/or as a result of mutation within the individual, can result in nonfunctional or partially functional TRF2, which in turn leads to dysregulation of iron distribution and resulting inflammation. Such mutations can occur within the structural portion of the gene that encodes for the TRF2 protein, regulatory regions that modulate transcription of the structural portion of the gene, and/or portions of the gene that signal the beginning or end of translation of the gene transcript.

Embodiments of the inventive concept include compositions and methods that correct such mutations and improve or restore expression of functional TRF2 protein in an individual having a mutation in an endogenous TRF2 gene or a regulatory site associated with an endogenous TRF2 gene. In some embodiments the mutated endogenous TRF2 gene or regulatory site can be modified to return the mutated portion to a wild-type sequence.

Without wishing to be bound to this example, it should be appreciated that the human endogenous TRF2 gene is located on chromosome 7 (specifically, at site 7q22.1) and is recorded in the extant literature. This gene encodes a single-pass type II membrane protein that is a member of the transferrin receptor-like family. The encoded protein mediates cellular uptake of transferrin-bound iron, and may be involved in iron metabolism, hepatocyte function and erythrocyte differentiation. Mutations in this gene have been associated with hereditary hemochromatosis type III.

Any suitable gene editing method can be used for this purpose. For example, Clustered regularly interspaced short palindromic repeats (CRISPR)/cas9 gene editing (as described in Li et al. Nature Signal Transduction and Targeted Therapy (2023) 8:36) can be used to replace a segment of a TRF2 structural gene or a single amino acid containing a mutation (e.g., a point mutation\ resulting in the formation of a premature stop codon) to the wild-type sequence, permitting transcription of the entire gene. Similarly, such methods can be used to correct point mutations that result in the inclusion of an incorrect amino acid in the expressed protein that negatively impacts function (e.g., placement of a hydrophobic amino acid in a position occupied by a charged amino acid in the wild-type sequence, or insertion of proline rather than the wild-type amino acid). Such methods can also be used to insert additional codons to return deletion mutations to the wild-type sequence. Such methods can, for example, correct genetic disease related to iron transport, storage, and/or distribution (e.g., hemochromatosis) and/or inflammatory conditions that result from dysregulation of iron transport, storage, and/or distribution.

Alternatively, suitable methods (for example, CRISPR/cas9 gene editing or a recombinant retrovirus as described in Anson, Genet Vaccines Ther. 2004; 2:9) can be used to introduce a wild-type TRF2 structural gene into an individual's nuclear DNA or in the DNA of an organelle to provide a supplementary and functional endogenous TRF2 gene, expression of which can correct genetic disease related to iron transport, storage, and/or distribution (e.g., hemochromatosis) and/or inflammatory conditions that result from dysregulation of iron transport, storage, and/or distribution. Such CRISPR/cas9 gene editing methods can be implemented using a recombinant virus as a vector, for example a recombinant adenovirus.

The compositions and methods described above relate to treatment of inflammatory conditions by modulating iron transportation and/or distribution mechanisms to alleviate dysregulation of iron transport and/or distribution. The Inventor contemplates that such inflammatory conditions can also or additionally be alleviated or their impact on the individual reduced by addressing ancillary or secondary effects of dysregulation of iron transport and/or distribution.

Iron is a cofactor of cytochrome C oxidase, an enzyme that can act as the terminal complex IV portion of the mitochondrial electron transport chain. Cytochrome C oxidase acts to transfer electrons provided by other components of the electron transport chain to molecular oxygen (O₂), resulting in the formation of water (H₂O). Without the necessary iron cofactor cytochrome C oxidase activity within mitochondria can be reduced, leading to the formation of potentially harmful species. Among these is superoxide anion (O₂⁻), which results from partial reduction of O₂. The superoxide anion is extremely reactive, and can directly damage proteins and nucleic acids within a cell or react to form other reactive species that are similarly destructive. Such damage can cause or contribute to inflammatory diseases and conditions. Such partial reduction of oxygen can be a result of reduced cytochrome C oxidase activity, which in turn can be a result of dysregulation of the transportation and/or distribution of iron.

Superoxide dismutase (SOD) is an enzyme that catalyzes the reaction of superoxide with water to form hydrogen peroxide (H₂O₂), which is less reactive with biomolecules than superoxide, and molecular oxygen. SOD is normally expressed as a family of enzymes with different distributions within the body. For example, SOD1 is encoded by a gene located on chromosome 21 (specifically at 21q22.1) and is found in the cytoplasm of the cell. SOD2 is located on chromosome 6 (specifically at 6925.3), and is found within mitochondria of the cell. SOD3 is found on chromosome 4 (specifically at 4p15.3-p15.1), and is found outside of the cell. Within the context of this application SOD can refer to any of these forms. In preferred embodiments SOD refers to SOD2.

Superoxide dismutase is normally expressed at levels appropriate for processing superoxide generated during normal activity of the electron transport chain, however such normal levels of SOD expression may not be sufficient to adequately process elevated levels of superoxide resulting from dysregulation of iron transport and/or distribution. The Inventor contemplates that compositions and methods as described above, therefore, can be complemented by modulating SOD content of cells (e.g., cells of an individual suffering from dysregulation of iron transport and/or distribution). In some embodiments such modulation of SOD content of a cell can be complementary to modulation of TRF2 and/or hepcidin. In other embodiments modulation of SOD content of a cell can be utilized without modulation of TRF2 and/or hepcidin.

SOD content of a cell of an individual in need of treatment for an inflammatory condition (e.g., an inflammatory condition resulting from dysregulation of iron transport and/or distribution) can be modulated (e.g., increased over normal levels of endogenous SOD expression) by any suitable means. For example, mutations in an endogenous SOD gene (e.g., that result in reduced or abnormal activity, transcription, and/or translation can be modulated by correcting the mutation using CRISPR/cas9 gene editing and/or introduction of a functional exogenous SOD gene using a viral vector (e.g., an adenovirus, a retrovirus, etc.). In some embodiments SOD protein can be introduced directly into a cell, for example through liposomal packaging or encapsulation within a micelle. Modulation of SOD as described above can be combined with modulation of TRF2, modulation of hepcidin, or modulation of both TRF2 and hepcidin.

It should be appreciated that the presence of excessive levels of superoxide can also result in dysregulation of calcium balance between the interior and exterior of the cell (see Baev et al. Cells. 2022 February; 11 (4): 706). Without wishing to be bound by theory, the Inventor believes that this is a result of dysregulation of calcium transport proteins (e.g., NCX, PMCA etc.) as a result of dysregulation of iron transport and/or distribution. Further, such dysregulation of calcium balance can cause or exacerbate and inflammatory disease and/or condition (e.g., inflammatory disease and/or condition resulting from dysregulation of iron transport and/or distribution). Such dysregulation can include downregulation of transcription and/or expression of calcium transport proteins, for example as a result on elevated extracellular calcium concentrations and intracellular regulatory mechanisms attempting to achieve normal homeostasis. Further, the Inventor believes that such elevated extracellular calcium concentration can interfere with transport of iron into the cell, causing or exacerbating an imbalance between intracellular and extracellular iron concentrations.

Such an imbalance between extracellular and intracellular calcium concentration can be corrected by modulation of calcium transport protein activity, expression, and/or transcription (e.g., upregulation) of calcium transport proteins. Examples of suitable calcium transport proteins include, but are not limited to, Transient Receptor Potential Canonical 1 (TRPC1), Voltage Dependent Calcium Channels (e.g., CaV1.1, CaV1.2, CaV1.3, CaV1.4), Plasma Membrane Calcium ATPase (PMCA), and/or Sodium-Calcium Exchanger (NCX). Activity and/or expression of such calcium transport proteins can be modulated by any suitable means. For example, mutations in an endogenous calcium transport protein gene (e.g., that result in reduced or abnormal activity, transcription, and/or translation can be modulated by correcting the mutation using CRISPR/cas9 gene editing and/or introduction of a functional exogenous calcium transport protein gene using a viral vector (e.g., an adenovirus, a retrovirus, etc.). In some embodiments calcium transport protein can be introduced directly into a cell, for example through liposomal packaging or encapsulation within a micelle. Modulation of a calcium transport protein as described above can be used in combination with modulation of TRF2, modulation of hepcidin, modulation of SOD, or any combination of these.

Alternatively, inflammatory diseases or conditions resulting from dysregulation of iron transport and/or distribution and amenable to treatment by modulation of calcium can be treated by modulating the balance between extracellular and intracellular calcium by administering compounds that can modulate existing mechanisms for calcium homeostasis. Examples of suitable compounds can include calcium channel blockers. Suitable calcium channel blockers include, but are not limited to Amlodipine (Norvasc), Diltiazem (Cardizem, Tiazac), Felodipine, Isradipine, Nicardipine, Nifedipine (Procardia), Nisoldipine (Sular), and Verapamil (Verelan). Alternatively or in addition, calcium balance can be modulated by administration of a calcium supplement and/or by administration of vitamin D. Such methods can be used in combination with modulation of TRF2, modulation of hepcidin, modulation of SOD, modulation of a calcium transport protein, or any combination of these.

In some embodiments, methods of the inventive concept can also incorporate one or more supplemental iron removal or complexation therapy (ies) in a treatment plan for an individual to be treated. Such a supplemental iron removal therapy can be provided prior to, during, or after administration of compositions as described above. Suitable supplemental iron removal or complexation therapies include phlebotomy and/or chelation therapy. The Inventor believes that synergistic (i.e., greater than additive) effects are observed in regard to reduction in symptoms and/or improvement in disease state on combined use of the compositions described above and a supplemental iron removal or complexation therapy.

Methods of the inventive concept can be implemented by any suitable means. Contemplated methods include, but are not limited to infusion, intravascular injection, subcutaneous injection, intramuscular injection, intraperitoneal injection, inhalation (e.g., of a mist, powder, or aerosol suspension), topical application (e.g., to skin and/or mucous membrane of an individual), ingestion, and introduction to the lower gastrointestinal tract (e.g., via a suppository and/or enema) of a composition that includes a therapeutic composition as described above. Such treatment can be provided as a single treatment that is effective to eliminate symptoms or reduce them to an acceptable level, or can include multiple treatments until a satisfactory degree of improvement is achieved. Such treatment can be provided every hour, every 2 hours, every 4 hours, every 6 hours, 3 times a day, 2 times a day, once a day, once every 2 days, 3 times a week, 2 times a week, weekly, every 2 weeks, every 3 weeks, every 4 weeks, once a month, every 2 months, every 3 months, 3 times a year, 2 times a year, annually, every 18 months, every 2 years, every 3 years, every 4 years, every 5 years, or at longer intervals. In some embodiments the manner of treatment (i.e., the nature of the modification to a cell), the mode of application, and/or the interval between treatments can be changed over the course of treatment. In some embodiments treatment may be required for the remainder of an individual's lifetime.

In some embodiments of the inventive concept compositions and/or methods as described above can be applied directly to modify cells within an individual, providing in vivo treatment. In some embodiments of the inventive concept compositions and/or methods as described above can be applied to a cell or cells extracorporeally, for example under in vitro conditions. Such modified cells can then be introduced into an individual to be treated, for example by infusion, injection, and/or implantation. Cells utilized in such in vitro methods can be obtained from the individual to be treated (i.e., autologous cells). In some embodiments such cells can be allogenic cells obtained from another source, such as a donor or a cell bank. In some embodiments both autologous and allogenic cells can be used in such in vitro methods. In such embodiments autologous and allogenic cells can be modified separately and applied separately, modified separately and mixed prior to application, or modified in a common in vitro environment (e.g., within the same culture flask).

Such in vitro methods can include steps of characterizing cells that have been modified prior to introduction into the individual to be treated. Any suitable method for characterization can be used. Suitable methods include, but are not limited to, cell sorting (such as fluorescence activated cell sorting or FACS). In such embodiments one or more populations of modified cells (e.g., cells showing a degree of modification that meets or exceeds a predetermined value, such as level of expression and/or co-expression of different proteins) can be isolated and utilized for treatment.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method of treating an inflammatory disease or iron transport dysregulation resulting from inflammation, comprising:
identifying an individual in need of treatment for an inflammatory disease associated with dysregulation of iron metabolism or iron transport dysregulation resulting from inflammation, wherein an endogenous TRF2 gene of the patient comprises a mutation, wherein the mutation decreases expression or function of the endogenous TRF2 gene; and
modifying a cell of the individual by modifying a nucleotide sequence at the mutation.

2. The method of claim 1, wherein the mutation is a point mutation, and wherein modifying the nucleotide sequence comprises replacement of a nucleotide base at the point mutation.

3. The method of claim 1, wherein the mutation comprises a deletion, and wherein modifying the nucleotide sequence comprises insertion of a wild type nucleotide sequence at the deletion.

4. The method of claim 1, wherein the mutation occurs within a structural portion of the TRF2 gene.

5. The method of claim 1, wherein the mutation occurs within a regulatory portion of the TRF2 gene.

6. The method of claim 1, wherein modifying the cell is performed using a gene editing method.

7. The method of claim 6, wherein the gene editing method is Clustered regularly interspaced short palindromic repeats (CRISPR)-cas9 gene editing.

8. The method of claim 1, further comprising modulation of a superoxide dismutase (SOD) or a calcium transport protein of the cell.

9. A composition for treating an inflammatory disease or dysregulation of iron transport resulting from inflammation, comprising a micelle or vesicle enclosing TRF2, wherein TRF2 is selected from the group consisting of: (a) αTFR2 protein or an active fragment thereof and (b) βTFR2 protein or an active fragment thereof.

10. The composition of claim 9, further comprising an SOD protein or a calcium transport channel protein.

11. A method of treating an inflammatory disease or iron transport dysregulation resulting from inflammation, comprising:
identifying an individual in need of treatment for an inflammatory disease associated with dysregulation of iron metabolism or iron transport dysregulation resulting from inflammation; and
modifying a cell of the individual to up-regulate expression of hepcidin, wherein modifying the cell comprises introducing a first expression vector that encodes hepcidin or a factor that interacts with a regulatory element associated with a gene encoding hepcidin.

12. The method of claim 11, wherein modifying the cell comprises introducing a virus comprising the first expression vector.

13. The method of claim 11, further comprising modulation of a superoxide dismutase (SOD) or a calcium transport protein of the cell.

14. A method of treating an inflammatory disease or iron transport dysregulation resulting from inflammation, comprising:
identifying an individual in need of treatment for an inflammatory disease associated with dysregulation of iron metabolism or iron transport dysregulation resulting from inflammation, wherein an endogenous TRF2 gene of the patient comprises a mutation, wherein the mutation decreases expression or function of the endogenous TRF2 gene; and
modifying a cell of the individual by inserting an exogenous TRF2 gene into the individual's genome.

15. The method of claim 14, wherein the mutation is selected from the group consisting of a point mutation, a deletion, and an insertion.

16. The method of claim 14, wherein the mutation occurs within a structural portion of the endogenous TRF2 gene.

17. The method of claim 14, wherein the mutation occurs within a regulatory portion of the endogenous TRF2 gene.

18. The method of claim 14, wherein modifying the cell is performed using a retrovirus, wherein the retrovirus, wherein the retrovirus comprises the exogenous TRF2 gene.

19. The method of claim 14, further comprising modifying the cell by introducing TRF2 or a second expression vector encoding TRF2 into the cell.

20. The method of claim 14, further comprising providing a supplemental iron removal therapy to the individual.

21. The method of claim 14, further comprising modulation of a superoxide dismutase (SOD) or a calcium transport protein of the cell.
